(19) United States
(12) Patent Application Publication
(10) Pub. No.: US 2025/0302799 A1
(43) Pub. Date: Oct. 2, 2025

(54) PHARMACEUTICAL COMPOSITION CONTAINING FRAXETIN FOR PREVENTION OR TREATMENT OF ENDOMETRIOSIS

(71) Applicants: Research & Business Foundation Sungkyunkwan University, Suwon-si (KR); INDUSTRY-ACADEMIC COOPERATION FOUNDATION GYEONGSANG NATIONAL UNIVERSITY, Jinju-si (KR); Korea University Research and Business Foundation, Seoul (KR); SEOUL NATIONAL UNIVERSITY HOSPITAL, Seoul (KR)

(72) Inventors: Whasun LIM, Suwon-si (KR); Jisoo SONG, Icheon-si (KR); Gwonhwa SONG, Cheonan-si (KR); Wonhyoung PARK, Suncheon-si (KR); Jiyeon HAM, Daejeon (KR); Hee Seung KIM, Seoul (KR); Soo Jin PARK, Seoul (KR); Sunwoo PARK, Jinju-si (KR)

(73) Assignees: Research & Business Foundation Sungkyunkwan University, Suwon-si (KR); INDUSTRY-ACADEMIC COOPERATION FOUNDATION GYEONGSANG NATIONAL UNIVERSITY, Jinju-si (KR); Korea University Research and Business Foundation, Seoul (KR); SEOUL NATIONAL UNIVERSITY HOSPITAL, Seoul (KR)

(21) Appl. No.: 18/963,289

(22) Filed: Nov. 27, 2024

(30) Foreign Application Priority Data
Mar. 26, 2024 (KR) 10-2024-0041229

(51) Int. Cl. A61K 31/37; A61K 31/145; A61K 31/57; A61P 15/00

(52) U.S. Cl. CPC A61K 31/37; A61K 31/145; A61K 31/57; A61P 15/00

(57) ABSTRACT

Disclosed herein is a pharmaceutical composition for preventing or treating endometriosis, comprising fraxetin. When used as a pharmaceutical composition for preventing or treating endometriosis and complications associated therewith, with fraxetin or a pharmaceutically acceptable salt thereof as an active ingredient, the present disclosure offers effective management of endometriosis with fewer side effects compared to surgery and hormone therapy and thus can be advantageously used as a therapeutic agent for endometriosis and complications associated therewith.

### CROSS-REFERENCE TO RELATED APPLICATION(S)

This application is based on and claims priority under 35 U.S.C. 119 to Korean Patent Application No. 10-2024-00041229, filed on Mar. 26, 2024, in the Korean Intellectual Property Office, the disclosure of which is herein incorporated by reference in its entirety.

### BACKGROUND OF THE INVENTION

### 1. Field of the Invention

The present disclosure was conducted with the support of the Ministry of Health and Welfare of the Republic of Korea under Project ID No. 1465039775 and Project No. HI22C1424000023. The managing institution for this project is the Korea Health Industry Development Institute, and the research project title is “Public Health Medical Technology Research Project”, with a specific focus on “Research for Clinical Application Based on GPCR Target Discovery for Inflammasome Modulation and Safety Validation to Overcome Infertility Linked to Endometriosis”. The host institution is the Sungkyunkwan University Research & Business Foundation, and the research period is from Jan. 1, 2023, to Dec. 31, 2023.

This patent application claims the benefit of and priority to Korean Patent Application No. 10-2024-00041229, filed with the Korean Intellectual Property Office on Mar. 26, 2024, the disclosure of which is incorporated herein by reference.

The present disclosure pertains to a pharmaceutical composition containing fraxetin for the prevention and treatment of endometriosis.

### 2. Description of the Prior Art

Endometriosis is a chronic gynecological condition

that affects 5-10% of women of reproductive age. It is characterized by the presence of endometriosis-like tissue outside the female reproductive organs. Major symptoms of endometriosis include pelvic pain, dysmenorrhea, and pain during defecation. These symptoms significantly impact the quality of life of patients, and about half of endometriosis patients face infertility. Recently, the prevalence of endometriosis has been increasing among women of reproductive age, yet its exact pathogenesis remains unclear, making precise diagnosis challenging.

Therapeutic approaches for treating endometriosis include surgery, hormone therapy, and anti-inflammatory treatments, mainly targeting symptom relief. However, current endometriosis treatments are associated with high recurrence rates and side effects, such as bone density loss, amenorrhea, weight gain, and depression, which limits their effectiveness.

Accordingly, there is a need for treatments with fewer side effects that can effectively manage endometriosis and improve the quality of life for patients. Consequently, research on phytochemicals exhibiting various physiological effects has recently increased.

Fraxetin, a coumarin-derived substance from Fraxinus rhynchophylla (ash tree), is known for its anti-proliferative, antifungal, antioxidant, and anti-inflammatory properties. Studies also suggest that it inhibits cell proliferation in ovarian, breast, liver, and colorectal cancers, induces apoptosis, and shows anti-cancer effects by disrupting mitochondrial function. However, the effect of fraxetin on endometriosis, a condition affecting women's health, has not yet been established.

Through research on plant-derived components that provide significant effects in preventing, improving, or treating endometriosis, the inventors confirmed that fraxetin, isolated from Fraxinus rhynchophylla, inhibits cell proliferation and migration in endometrial cells, induces cell cycle arrest, and promotes apoptosis. Furthermore, it was observed that fraxetin increases mitochondrial membrane depolarization and the accumulation of calcium ions within cells and mitochondria. These findings suggest that fraxetin may serve as a potential therapeutic or adjuvant treatment for preventing or treating endometriosis, thereby culminating in the present disclosure.

### PRIOR ART DOCUMENTS

### Patent Literature

### Prior Art Documents

(Patent Literature 001) U.S. Patent No. US 2023/0089351 A (Published on Mar. 23, 2023)

### SUMMARY OF THE INVENTION

Leading to the present disclosure, intensive and thorough research conducted by the present inventors with the aim of discovering plant-derived components that not only alleviate the symptoms of endometriosis but also inhibit the progression of the disease and offers significant therapeutic and preventive effects and developing a pharmaceutical composition containing same, resulted in the finding that fraxetin, isolated from Fraxinus rhynchophylla (ash tree), exhibits effects in inhibiting cell proliferation and migration in endometrial cells, inducing cell cycle arrest, and promoting apoptosis and increases mitochondrial membrane depolarization and enhances the accumulation of calcium ions within cells and mitochondria, thus making it possible to use fraxetin as a potential therapeutic or adjuvant agent for preventing or treating endometriosis.

Accordingly, the present disclosure is to provide a pharmaceutical composition including fraxetin or a pharmaceutically acceptable salt thereof as an active ingredient for the prevention or treatment of endometriosis and complications associated with endometriosis.

Also, the present disclosure is to provide a food composition including fraxetin or a pharmaceutically acceptable salt thereof as an active ingredient for the prevention or alleviation of endometriosis and complications associated with endometriosis.

The present inventors have endeavored to discover plant-derived components that not only alleviate the symptoms of endometriosis but also inhibit disease progression, offering significant therapeutic and preventive effects. Through this research, it was confirmed that fraxetin, isolated from Fraxinus rhynchophylla (ash tree), inhibits cell proliferation and migration in endometrial cells, induces cell cycle arrest, and promotes apoptosis. Additionally, was observed that fraxetin increases mitochondrial membrane depolarization and calcium ion accumulation within cells and mitochondria. These findings in suggest that fraxetin may serve as a potential therapeutic or adjuvant agent for preventing or treating endometriosis, thereby culminating in the present disclosure. In an aspect thereof, the present disclosure provides

a pharmaceutical composition including fraxetin or a pharmaceutically acceptable salt thereof as an active ingredient for preventing or treating endometriosis and complications associated therewith.

In another aspect thereof, the present disclosure provides a method for preventing or treating endometriosis and complications associated therewith, the method including a step of administering fraxetin or a pharmaceutically acceptable salt thereof to a subject in need.

The method for preventing or treating endometriosis and complications associated therewith utilizes fraxetin or a pharmaceutically acceptable salt thereof as the active ingredient in the pharmaceutical composition mentioned above. To prevent redundancy, details common to both aspects of the present disclosure are equally applicable and are omitted from this specification.

Herein, “fraxetin” is one of the coumarin-derived substances from Fraxinus rhynchophylla, represented by the following Chemical Formula 1:

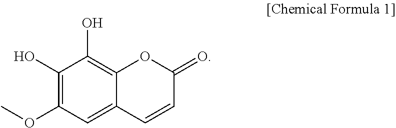

In the present disclosure, Fraxinus rhynchophylla is a deciduous tree of the family Oleaceae, widely distributed in East Asia, including Korea, China, and Japan.

Reported components of Fraxinus rhynchophylla include esculetin, esculin, and tannins, which are known for their anti-inflammatory and pain-relieving properties. Therefore, Fraxinus rhynchophylla is reported to have positive effects on conditions such as gastritis, asthma, and bronchitis, support liver health, and benefit eye health, making it effective against eye conditions such as glaucoma, cataracts, and hemorrhage. In traditional Korean medicine, it is used to treat conditions like rheumatism, gout, enteritis, and leucorrhea due to its analgesic, anti-inflammatory, and antipyretic effects.

The fraxetin of the present disclosure can be used in the form of a pharmaceutically acceptable salt derived from an inorganic or organic acid. Preferred salts include one or more selected from the group consisting of hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methane sulfonic acid, benzene sulfonic acid, and toluene sulfonic acid, without being limited thereto.

In addition to fraxetin or a pharmaceutically

acceptable salt thereof, the present disclosure may include hydrates, solvates, and optical isomers that can be prepared therefrom.

The term “hydrate” as used herein refers to a compound of the present disclosure or salts thereof containing a stoichiometric or non-stoichiometric amount of water bound by non-covalent intermolecular forces. The hydrate of the compound represented by Chemical Formula 1 may include a stoichiometric or non-stoichiometric amount of water bound by non-covalent intermolecular forces. This hydrate can contain one or more equivalents of water, preferably 1 to 5 equivalents. Such hydrates can be prepared by crystallizing the compound of Chemical Formula 1, isomers thereof, or pharmaceutically acceptable salts thereof from water or a solvent containing water.

The term “solvate” as used herein refers to a compound of the present invention or salts thereof containing a stoichiometric or non-stoichiometric amount of a solvent bound by non-covalent intermolecular forces. Suitable solvents include those that are volatile, non-toxic, and/or suitable for human administration.

As used herein, the term “isomer” refers to a compound of the present invention or salts thereof with the same chemical or molecular formula but differing in structure or stereochemistry. Such isomers include structural isomers such as tautomers, stereoisomers like R or S enantiomers having asymmetric carbon centers, geometric isomers (trans, cis), and optical isomers (enantiomers). All these isomers and their mixtures are also included within the scope of the present disclosure.

The term “including as an active ingredient” in the present disclosure refers to including a sufficient amount of fraxetin to achieve its pharmacological efficacy or activity, with the possibility of additional components added for drug delivery, stabilization, and formulation.

The term “endometriosis” as used in the present disclosure refers to a condition in which endometrial tissue proliferates outside the uterine cavity surface. Endometriosis occurs in approximately 10-15% of women of reproductive age and is a significant cause of pelvic inflammatory disease, pelvic adhesions, ovarian cysts, uterine fibroids, ectopic pregnancy, and infertility. The pathophysiology of endometriosis reveals a similarity to tumor cells in terms of growth and development, as endometrial tissue invades other tissues or migrates beyond its original location, resulting in distant metastasis.

Endometriosis presents a wide range of symptoms depending on the location of the lesions, affected organs, and extent of the lesions, making it a difficult disease to diagnose. Current clinical tests for diagnosing endometriosis: include transvaginal ultrasound, magnetic resonance imaging (MRI), blood tests, and laparoscopic surgery. Recently, various diagnostic methods have been developed, such as utilizing genetic markers to diagnose endometriosis or predict recurrence after treatment by analyzing the genetic characteristics of individual patients.

Endometriosis primarily occurs in the abdominal organs or peritoneum but can also affect other distant organs within the body, such as the intestines or lungs. Endometriosis occurring in the ovaries is referred to as an “endometrioma”. When endometriosis is present in the ovaries, the risk of ovarian cancer is higher than in the general population.

As used herein, the term “prevention” refers to all protective actions that inhibit or delay the onset of endometriosis, complications thereof, or at least one of its symptoms through the administration of the pharmaceutical composition of the present disclosure. It also includes treating individuals showing signs of improvement to prevent or inhibit recurrence of the disease.

The term “treatment” refers to all actions that improve or beneficially modify the symptoms of endometriosis, complications thereof, or at least one of the symptoms, including alleviation, reduction, or elimination of symptoms through administration of the pharmaceutical composition of the present disclosure.

The term “pharmaceutical composition”, as used herein, refers to a composition administered for a specific purpose and, in the context of this disclosure, is administered to prevent or treat endometriosis, its complications, or at least one of its symptoms. The pharmaceutical composition contains a pharmaceutically effective amount of fraxetin or a pharmaceutically acceptable salt thereof.

The pharmaceutical composition of the present disclosure can be formulated as powder, granules, tablets, coated tablets, pills, sugar-coated tablets, capsules, solutions, suspensions, gels, syrups, slurries, suppositories, enemas, emulsions, pastes, ointments, creams, lotions, powders, sprays, or suspensions.

The pharmaceutical composition of the present disclosure may additionally include appropriate carriers, excipients, or diluents commonly used in the manufacture of pharmaceutical compositions. Examples include lactose, dextrose, sucrose, sorbitol, mannitol, xylitol, erythritol, mannitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, or mineral oil.

The pharmaceutical composition of the present disclosure may further contain excipients, stabilizers, diluents, lubricants, wetting agents, sweeteners, flavoring agents, emulsifiers, suspending agents, preservatives, etc. For details of suitable pharmaceutically acceptable carriers, vehicles, excipients, stabilizers, or diluents, reference may be made to “Remington's Pharmaceutical Sciences” (19^{th} ed., 1995).

The pharmaceutical composition according to the present disclosure is administered in a pharmaceutically effective amount.

As used herein, the term “pharmaceutically effective amount” refers to an amount sufficient to achieve preventive, mitigating, or therapeutic efficacy for endometriosis, its complications, or at least one of its symptoms. The effective dose may vary depending on factors such as the type and severity of the patient's condition, drug activity, drug sensitivity, administration time, route of administration, excretion rate, treatment duration, and concurrent medications, among other factors well-known in the medical field. The composition's dosage can vary depending on the patient's age and weight but may be administered once or multiple times daily to achieve a therapeutic concentration in the blood effective for treating endometriosis.

The pharmaceutical composition of the present disclosure may be manufactured as a unit dosage form by formulating with pharmaceutically acceptable carriers and/or excipients or packaged in a multi-dose container according to methods readily implementable by those skilled in the field. The formulation may be in the form of solutions, suspensions, or emulsions in oil or aqueous media; or as extracts, powders, granules, tablets, or capsules, and may additionally contain dispersants or stabilizers.

The dosage of the composition can be adjusted based on factors like route of administration, disease severity, body weight, age, etc. Therefore, the dosage does not in any way limit the scope of the present disclosure.

The pharmaceutical composition of the present disclosure can be administered to a subject through various routes. All modes of administration may be contemplated, including intracranial, oral, subcutaneous, intraperitoneal, intravenous, intramuscular, intraspinal (intrathecal), sublingual, buccal, intrarectal, intravaginal, ear, intranasal, inhalation, spray via mouth or nose, cutaneous, and transdermal administration.

The pharmaceutical composition of the present disclosure can be administered as an individual therapy or combination with other therapies, administered in sequentially or simultaneously with conventional therapies, as single or multiple doses. Considering all the above factors, it is essential to administer the minimal amount needed to achieve the maximum effect without side effects, which can be easily determined by those skilled in the art.

In an embodiment of the present disclosure, fraxetin or a pharmaceutically acceptable salt thereof inhibits the proliferation of endometrial cells.

In this disclosure, endometrial cells may include cells derived from the vaginal mucosa, uterus, cervix, and endocervix.

Specifically, the endometrial cells may be VK2/E6E7 cell lines derived from the vaginal mucosa or End1/E6E7 cell lines derived from the uterus, cervix, and endocervix.

In an embodiment, fraxetin in the present disclosure was confirmed to inhibit the proliferation of endometrial cells by inducing cell cycle arrest in endometrial cell lines.

In another embodiment, fraxetin or a pharmaceutically acceptable salt thereof induces mitochondrial dysfunction in endometrial cells.

The term “mitochondrial dysfunction”, as used herein, refers to the loss of biological function of normal mitochondria, including loss of electron transport chain efficiency, decreased synthesis of high-energy molecules like ATP, increased production of reactive oxygen species (ROS), disruption of cellular respiration, reduction of mitochondrial membrane potential, mitochondrial fragmentation, and mutations in mitochondrial DNA, but with no limitations thereto.

In an embodiment, mitochondrial dysfunction includes one or more of the following: (a) depolarization of mitochondrial membrane potential; (b) calcium ion homeostasis disruption; (c) reduced ATP-related cellular respiration; or (d) combinations thereof.

In an embodiment, fraxetin in the present disclosure was confirmed to induce mitochondrial dysfunction by reducing membrane potential, resulting in mitochondrial depolarization and abnormal accumulation of calcium ions in matrix, which disrupts the cytoplasm and mitochondrial calcium ion homeostasis and inhibits cellular respiration, including basal respiration, maximal respiration, and ATP production in endometriosis cells.

The term “mitochondrial membrane potential” (ΔΨm), as used herein, refers to the potential generated by proton pumps essential for the energy storage process in oxidative phosphorylation. Mitochondrial membrane potential can be measured by commonly known methods in the field, such as JC-1, Rhodamine B hexyl ester, MitoTracker Red CMXRos, Rhodamine 6G, DiS-C3 (3), HRB & HR101, or TMRE (tetramethylrhodamine, methyl ester) staining methods, but is not limited thereto.

In this disclosure, the intracellular electrochemical potential of calcium ions plays a role in regulating various cellular signaling pathways through various ion channels, Specifically, calcium ion signaling pumps, and exchangers. is closely related to the production of reactive oxygen species (ROS), which regulate cell proliferation and apoptosis. Disrupted calcium ion homeostasis not only causes severe oxidative stress but also imposes calcium ion stress on mitochondria, which act as calcium reservoirs, adversely affecting mitochondria and mitochondrial membranes. Therefore, calcium ion homeostasis disruption leads to significant mitochondrial dysfunction by destabilizing the mitochondrial outer membrane.

In an embodiment of the present disclosure, fraxetin or a pharmaceutically acceptable salt thereof increases endoplasmic reticulum (ER) stress in endometrial cells.

In this disclosure, the endoplasmic reticulum plays a role in essential functions such as stress sensing, signaling, and regulating homeostasis of cellular organelles. ER stress is related to the release of calcium ions into the cytoplasm, where accumulated calcium ions inhibit autophagy and apoptosis pathways. The activation of IRE1A (also known as ERN1) induces ER-mediated mitochondrial apoptosis and inhibits the anti-apoptotic effect of BCL-2 through MAPK/JNK activation. Phosphorylated EIF2A suppresses protein translation and promotes the expression of GADD43, which is associated with growth inhibition.

In an embodiment, fraxetin was confirmed to increase intracellular stress by increasing the expression of proteins such as GRP78, p-EIF2A, and ATF6α.

In another embodiment of the present disclosure, the pharmaceutical composition modulates signaling pathways related to the proliferation of endometrial cells.

Specifically, the pharmaceutical composition is characterized by inhibiting PI3K/AKT and MAPK signaling pathways.

In the present disclosure, “phosphatidylinositol 4,5-bisphosphate” (PI3K) refers to a signaling molecule involved in various cellular functions such as cell cycle, cell motility, and apoptosis. PI3K is a lipid kinase that produces second messenger molecules that activate certain proteins, including serine/threonine kinases like PDK1 and AKT. PI3K is divided into three classes, with Class I including four different PI3Ks known as PI3Kα, PI3Kβ, PI3Kδ, and PI3Kγ.

In this disclosure, the “mitogen-activated protein kinase” (MAPK) signaling pathway is associated with cellular activities such as growth, differentiation, and stress response. It is a MAPK signaling cascade activated through phosphorylation of downstream regulatory proteins, including seven MAPKK homologs and four parallel MAPK pathways, with ERK1/ERK2, JNK, and p38 being well-known pathways.

According to an embodiment of the present disclosure, it was confirmed that fraxetin inhibits the proliferation of endometrial cells by modulating the phosphorylation patterns of AKT, P70, and S6 proteins in the PI3K/AKT signaling pathway, or of ERK1/2, p38, and JNK proteins in the MAPK signaling pathway.

In an embodiment, fraxetin was found to inhibit the development of endometriotic lesions by suppressing the expression of adhesion-related genes, increasing the expression of apoptosis-related genes, increasing the expression of endoplasmic reticulum stress-related genes, and decreasing the expression of mitochondrial function-related proteins in endometrial cell lines.

In an embodiment, fraxetin was found to inhibit the development of endometriosis by inducing cell death through mitochondrial dysfunction in endometrial cell lines.

In another embodiment, fraxetin was found to inhibit the proliferation of endometrial cell lines through tiRNA regulation and to induce cell death via mitochondrial dysfunction, thereby inhibiting the development of endometriosis.

In an embodiment of the present disclosure, the endometriosis-associated complications include one or more selected from the group consisting of pelvic inflammatory disease, pelvic adhesions, ovarian cysts, uterine fibroids, ectopic pregnancy, and infertility, but are not limited thereto.

In an embodiment of the present disclosure, the method includes administering a PI3K/AKT signaling pathway inhibitor, MAPK signaling pathway inhibitor, or GnRH agonist in addition to the administration of fraxetin to the subject in need.

As used herein, the term “signaling pathway inhibitor” refers to a substance that suppresses the expression or activity of signaling molecules involved in the growth, activation, and function of cells within endometriotic tissue.

In an embodiment of the present disclosure, the PI3K/AKT signaling pathway inhibitor may be wortmannin, although it is not limited to this compound.

In another embodiment of the present disclosure, the MAPK signaling pathway inhibitor may be U0126, although it is not limited to this compound.

In yet another embodiment of the present disclosure, the GnRH agonist may be dienogest, although it is not limited to this compound.

As described above, a pharmaceutical composition containing fraxetin according to the present disclosure may exhibit an inhibitory effect on cell proliferation in endometrial cell lines by fraxetin alone. However, a more enhanced cell proliferation inhibition effect can be expected when fraxetin is combined with existing signaling pathway inhibitors.

This combined treatment may involve administering the pharmaceutical composition as an individual therapy or in combination with other treatments, either sequentially or concurrently with conventional treatments.

The features and advantages of the present disclosure are summarized as follows:

(a) The present disclosure provides a pharmaceutical composition for the prevention or treatment of endometriosis and complications associated therewith, comprising fraxetin or a pharmaceutically acceptable salt thereof as an active ingredient.

(b) The present disclosure provides a health-functional food composition for the prevention or improvement of endometriosis and complications associated therewith, comprising fraxetin or a pharmaceutically acceptable salt thereof as an active ingredient.

(c) When using the pharmaceutical composition for the prevention or treatment of endometriosis and complications associated therewith, comprising fraxetin or a pharmaceutically acceptable salt thereof as an active ingredient, it can effectively manage endometriosis with fewer side effects compared to surgery and hormone therapy, making it a useful therapeutic agent for endometriosis and complications associated therewith.

### BRIEF DESCRIPTION OF THE DRAWINGS

The above and other aspects, features and advantages of the present disclosure will be more apparent from the following detailed description taken in conjunction with the accompanying drawings, in which:

FIGS. 1a to 1g illustrate the method of establishing an animal model of endometriosis and show the effects of fraxetin on endometriosis lesions in the animal model.

FIGS. 2a to 2f show the analysis results of mRNA expression of genes related to inflammation, adhesion, apoptosis, endoplasmic reticulum stress, and mitochondrial function in endometrial tissue treated with fraxetin;

FIGS. 3a to 3c show the analysis results of cell proliferation and cell cycle inhibition in endometrial cells treated with fraxetin;

FIG. 4 shows the analysis results of cell proliferation in normal endometrial cells treated with fraxetin.

FIGS. 5a to 5h show the analysis results of cell migration inhibition and MAPK and AKT signaling pathway inhibition in endometrial cells treated with fraxetin;

FIGS. 6a to 6f show the analysis results of changes in cell viability and target protein phosphorylation patterns in endometrial cells treated with fraxetin, MAPK inhibitor, AKT inhibitor alone, and in combination;

FIGS. 7a to 7f show the analysis results of changes in mitochondrial membrane potential, basal metabolic rate from mitochondrial respiration, ATP production, and reactive oxygen species in endometrial cells treated with fraxetin;

FIGS. 8a to 8i show the analysis results of changes in apoptosis and endoplasmic reticulum stress-related proteins in endometrial cells treated with fraxetin;

FIGS. 9a to 9d show the analysis results of reactive oxygen species production and apoptosis effects in endometrial cells treated with fraxetin and dienogest;

FIGS. 10a to 10k show the analysis results of tiRNA regulation patterns and the effects on apoptosis and mitochondrial membrane potential depolarization in endometrial cells treated with fraxetin and candidate tiRNAs; and

FIG. 11 is a schematic diagram showing the mechanisms of cell proliferation inhibition and apoptosis induction by fraxetin in endometrial cells.

### DETAILED DESCRIPTION OF THE EXEMPLARY EMBODIMENTS

Below, a better understanding of the present disclosure may be obtained through the following examples, which are set forth to illustrate, but are not to be construed to limit, the present disclosure.

### EXAMPLES

Throughout this specification, unless otherwise stated, the “%” used to indicate the concentration of a particular substance represents (weight/weight) % for solid/solid mixtures, (weight/volume) % for solid/liquid mixtures, and (volume/volume) % for liquid/liquid mixtures.

### Experimental Example 1: Animal Experiment

Seven-week-old female C57/BL6 mice were purchased from DBL (Eumseong, South Korea). After a 7-day acclimation period, they were subcutaneously injected with 2 mg/50 μL of progesterone two days before surgery. After anesthetizing the mice, the cervix was excised and autografted onto the peritoneum to induce endometriosis. After culturing for two weeks, the mice were randomly assigned to groups and administered either a vehicle as the control, fraxetin at 30mg/kg, or dienogest at 1 mg/kg orally for four weeks.

### Experimental Example 2: Histological Analysis

For histological analysis, tissues were lesion prepared as paraffin blocks and sectioned onto slides. Hematoxylin-eosin (H&E) staining was performed using Mayer's hemalum solution and a Prussian Blue staining kit (Cat. Ab150674, Abcam) histological analysis and for immunohistochemistry. Immunohistochemistry was conducted using an avidin-biotin complex kit (Cat. PK-4001, Vector Laboratories).

### Experimental Example 3: Cell Culture

The endometrial cell lines VK2/E6E7, End1/E6E7, and normal endometrial stromal cell line T-HESC were purchased from ATCC. The cells were cultured in keratinocyte serum-free medium or DMEM/F12 1:1 medium containing 10% FBS for monolayer culture. Endometrial cell lines (VK2/E6E7,End1/E6E7) and normal cells were cultured at 37° C. in a 5% CO₂ incubator.

### Experimental Example 4: Analysis of Fraxetin's Inhibition of Cell Proliferation Using BrdU

To examine the effects of fraxetin on cell proliferation, the inventors performed a BrdU cell proliferation assay.

First, 5×10³ cells of VK2/E6E7, End1/E6E7, and T-HESC were each distributed in 100 μL of medium in 96-well plates.

Fraxetin was then administered in a dose-dependent manner (0μM, 5 μM, 10 μM, 20 uM, 50 μM, 100 uM) and cultured for 48 hours, followed by the BrdU assay using a BrdU kit (Cat No: 1167229001, Roche) according to the manufacturer's instructions.

After 48 hours of incubation, 10 μM BrdU (Bromodeoxyuridine) was added to each well and incubated at 37° C. in a 5% CO₂ incubator for 2 hours. The cells were then fixed, reacted with anti-BrdU-POD solution at room temperature for 90 minutes, and washed three times. Finally, 100 μL of 3, 3′, 5, 5′-tetramethylbenzidine substrate was added, and absorbance was measured at wavelengths of 370 nm and 492 nm to analyze cell proliferation.

### Experimental Example 5: Cell Cycle Analysis

The inventors performed flow cytometry to examine the changes in the cell cycle of endometrial cells induced by fraxetin.

In brief, 5×10³ cells of VK2/E6E7, End1/E6E7, and T-HESC were treated with fraxetin in a dose-dependent manner (0 μM, 5 μM, 10 μM, 20 μM, 50 μM, 100 μM) and incubated for 48 hours at 37° C. in a 5% CO₂ incubator.

Afterward, cells were detached from the culture plates using trypsin, washed with 0.1% BSA/PBS, and fixed in 70% ethanol for 24 hours. The cells were then washed with 0.1% BSA/PBS, treated with RNase A (Sigma), and stained with PI. The stained solution transferred to FACS tubes, and fluorescence intensity and cell cycle were analyzed using a flow cytometer.

### Experimental Example 6: Cell Migration Analysis

To measure the effects of fraxetin on endometrial cell migration, the inventors used a migration culture dish insert from Ibidi to analyze cell mobility.

In brief, endometrial cells (VK2/E6E7, End1/E6E7) were placed in compartmentalized wells within the culture dish and cultured until they filled the compartments. Afterward, the insert separating the compartments was removed, creating a 500 μm gap for cell migration. The cells were then cultured with medium containing fraxetin at 37° C. in a 5% CO₂ incubator. Cell movement across the gap was observed using an optical microscope (DM3000, Leica) and photographed, and the migration distance was calculated and quantified.

### Experimental Example 7: Protein Expression Analysis

To analyze protein expression related to the development of endometriosis following fraxetin treatment, the inventors conducted western blot analysis.

Briefly, endometrial cells were treated with fraxetin in a dose-dependent manner or in combination with signaling pathway inhibitors. Total proteins were extracted from the treated cells, and protein quantification was performed using the Bradford protein assay (Bio-Rad, Hercules, CA, USA). The extracted proteins were then denatured at 95° C. for 5 minutes and subjected to electrophoresis on a 10% SDS/PAGE gel before being transferred to a nitrocellulose membrane. The membrane was subsequently incubated with primary and secondary antibodies, and target protein expression was analyzed using chemiluminescence detection reagents (Supersignal West Pico, Pierce, Rockford, IL, USA) with the ChemiDoc EQ system and Quantity One software (Bio-Rad).

### Experimental Example 8: Mitochondrial Membrane Potential Measurement Using JC-1 Staining

To examine changes in mitochondrial membrane potential (MMP) in endometrial cells induced by fraxetin, the inventors used a mitochondria staining kit (Cat no: CS0390, Sigma-Aldrich) to measure MMP.

In brief, End1/E6E7 and VK1/E6E7 endometrial cell lines were cultured in 6-well plates, treated with fraxetin in a dose-dependent manner (0 μM, 5 μM, 10 μM, 20 μM, 50 μM, 100 μM), and incubated at 37° C. in a 5% CO₂ incubator for 48 hours. Afterward, the cells were detached using trypsin, centrifuged to obtain cell pellets, resuspended in JC-1 staining solution, and incubated at 37° C. in a 5% CO₂ incubator for 20 minutes. The stained cells were centrifuged again, washed with 1X JC-1 staining buffer, and analyzed using flow cytometry to assess changes in mitochondrial membrane potential.

### Experimental Example 9: Mitochondrial Respiration Control Analysis

To confirm the effects of fraxetin on mitochondrial respiration in endometrial cells, the inventors used an XF Cell Mitostress kit.

In brief, endometrial cells were seeded in 24-well plates and cultured at 37° C. in a 5% CO₂ incubator for 16 hours, followed by treatment with 50 fraxetin. Subsequently, various mitochondrial respiration parameters were measured by sequential treatment with oligomycin, FCCP, rotenone, and antimycin A in the two cell lines.

### Experimental Example 10: Reactive Oxygen Species (ROS) Generation Analysis

To assess the inhibitory effect of fraxetin on ROS generation in endometrial cells, the inventors used flow cytometry to measure ROS levels.

In brief, endometriosis cells were stained with DCFH-DA (final concentration 10 μM) for 30 minutes and treated with 50 μM fraxetin in experimental groups, with or without 2 mM N-acetylcysteine (NAC; Cat No. A0737, Sigma), in a 37° C. incubator. Afterward, the cells were washed with PBS, and ROS levels were detected by measuring fluorescence DCF via flow cytometry.

### Experimental Example 11: Intracellular and Mitochondrial Calcium Concentration Analysis Using Fluo-4 and Rhod-2 Staining

To examine intracellular or mitochondrial calcium concentration changes in endometrial cells induced by fraxetin, the inventors conducted experiments using 3 μM fluo-4 acetoxymethyl ester (AM) (Invitrogen) and 3 μM rhod-2.

First, cells were cultured in 6-well plates, treated with fraxetin in a dose-dependent manner (0 μM, 5 μM, 10 μM, 20 μM, 50 μM, 100 μM), and incubated at 37° C. in a 5% CO₂ incubator for 48 hours. The cells were then detached using trypsin, washed with PBS, and stained with 3 μM fluo-4 or 3 μM rhod-2 for 20 minutes at 37° C. in a 5% CO₂ incubator. After staining, the cells were washed with PBS, transferred to FACS tubes, and analyzed by flow cytometry to measure intracellular or mitochondrial calcium concentrations.

### Experimental Example 12: Apoptosis Analysis Using Annexin V and Propidium Iodide Staining

To assess the apoptosis-inducing effect of fraxetin in endometrial cells, the inventors used a FITC Annexin V Apoptosis Detection Kit I (BD Sciences).

First, VK2/E6E7 and End1/E6E7 cells were cultured in 6-well plates, treated with fraxetin in a dose-dependent manner (0 μM, 5 M, 10 μM, 20 M, 50 μM, 100 μM), and incubated for 48 hours at 37° C. in a 5% CO₂ incubator. The cells were then detached using trypsin, washed with PBS, and mixed slowly with 1 mL of 1X binding buffer. After centrifugation, the cell pellet was resuspended in 200 μL of 1X binding buffer. The suspension (100 μL) was transferred to a 1.5 mL brown tube, and 5 μL of Annexin V and PI (propidium iodide) were added, followed by staining at room temperature for 15 minutes. After adding 400 μL of 1X binding buffer, the stained solution was transferred to a 5 mL FACS tube, and the number of apoptotic cells was measured by analyzing fluorescence intensity using flow cytometry.

### Experimental Example 13: tiRNA Expression Measurement and Target tiRNA Inhibitor Administration

To confirm the expression of tiRNA (tRNA-derived stress-induced RNAs) associated with the development of endometriosis, the inventors conducted real-time PCR.

First, information on candidate tiRNA sequences and mature tRNA sources was obtained from MINTbase (https://cm.jefferson.edu/MINTbase/). miRNA CDNA synthesis was performed using a cDNA kit (Agilent Technologies) to confirm tiRNA expression, with U6 snRNA used for normalization. For transfection of the tiRNA^{His GTG} inhibitor (5′-AAC CGA GAG UAC UAA CCA CUA UAC GAU CAC CGC-3′) (40 nM), Lipofectamine 2000 was used in Opti-MEM.

### Experimental Example 14: Statistical Analysis

All experimental results were analyzed using the SAS statistical analysis program, calculating means and standard errors, with one-way ANOVA performed for significance testing at the p<0.05 level.

### EXAMPLE 1: Analysis of Reductive Effect of Fraxetin on Endometriotic Lesion Growth

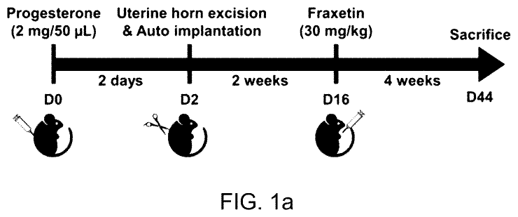

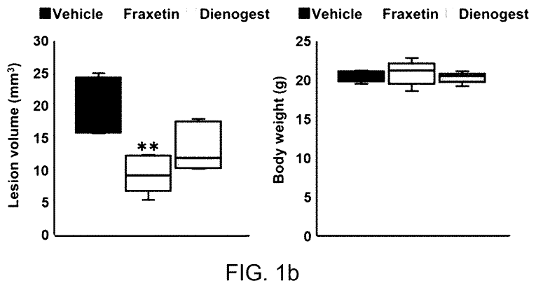

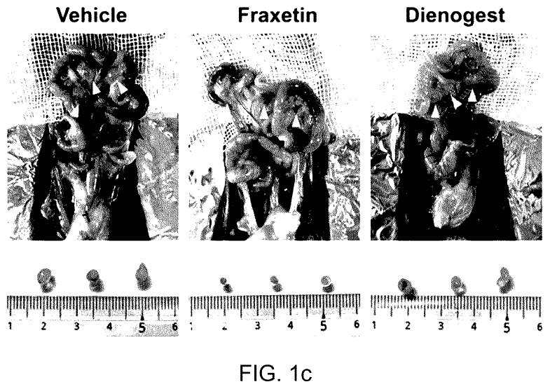

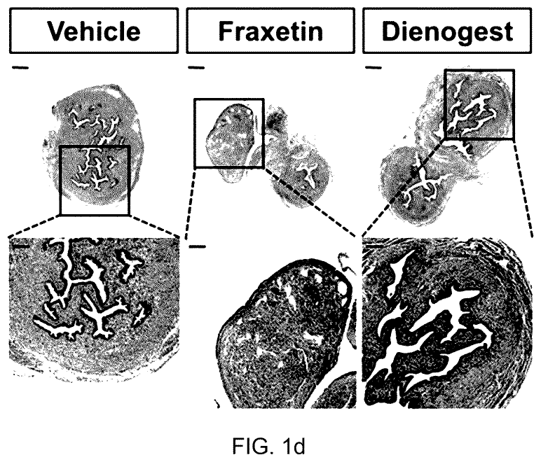

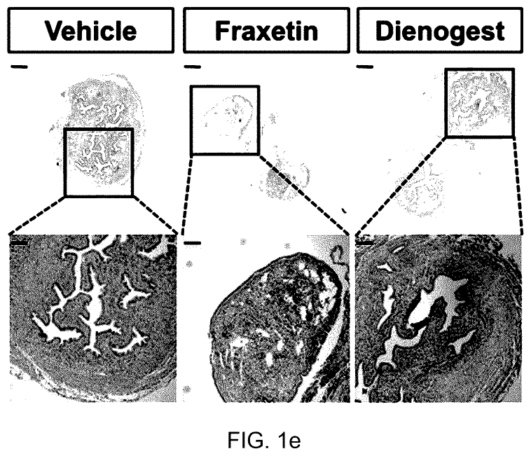

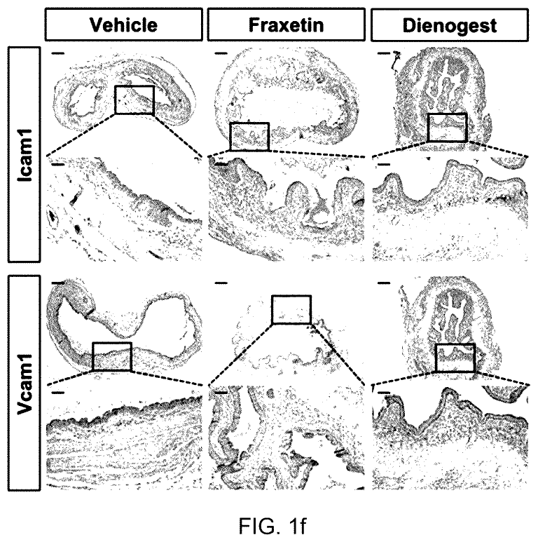

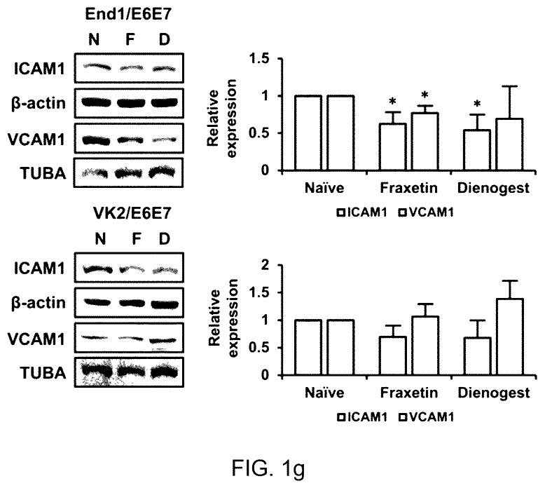

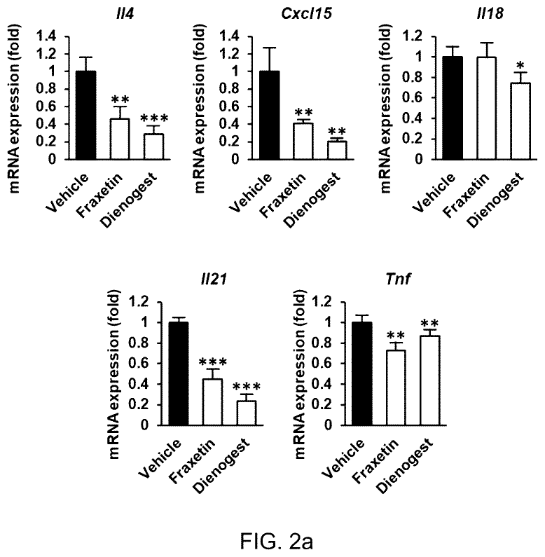

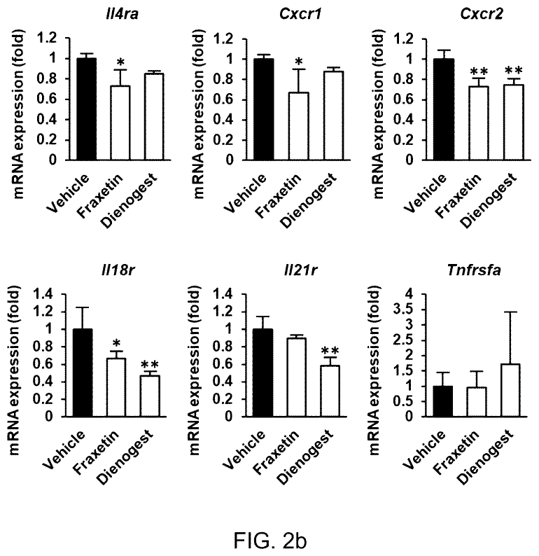

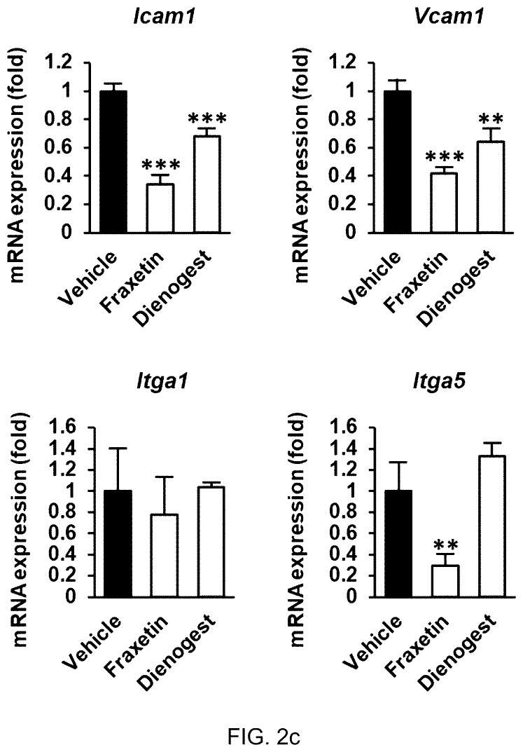

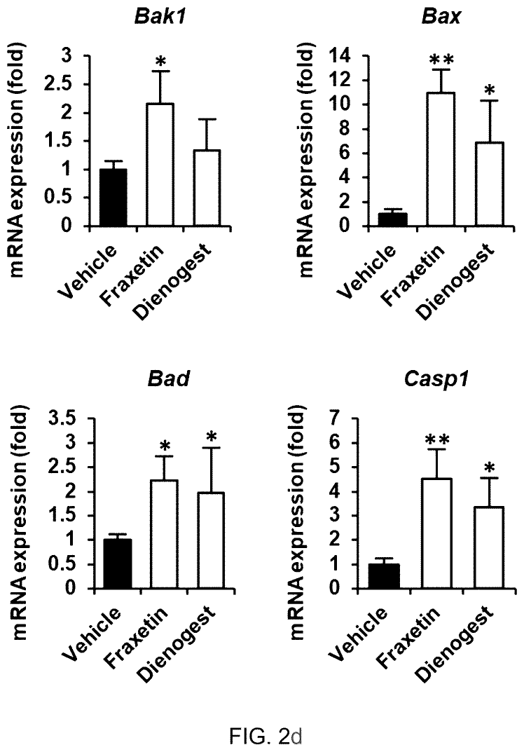

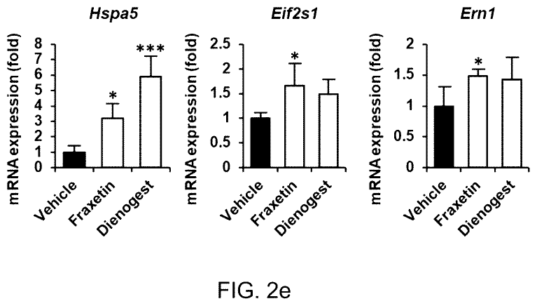

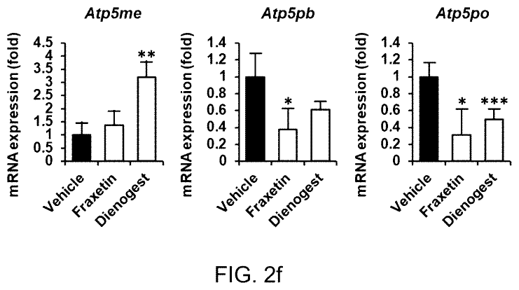

To investigate the effect of fraxetin on endometriotic lesions in an animal model, the inventors administered fraxetin and dienogest to the model and observed changes in lesion tissue (FIG. 1a), with results shown in FIGS. 1 and 2.

As shown in FIGS. 1b to 1e, fraxetin significantly reduced the size of endometriotic lesions. Observation of tissue after iron staining revealed an increase in immune cell infiltration in the fraxetin-treated group.

Additionally, as shown in FIG. 1f, expression of Icam1 and Vcaml within the lesion tissue was reduced in the fraxetin-treated group compared to the control group. A significant decrease in expression levels was also observed in human endometriosis cell lines VK2/E6E7 and End1/E6E7.

These results suggest that fraxetin may reduce endometriotic lesion growth by inhibiting inflammatory proliferation.

Furthermore, analysis of mRNA expression of inflammation-related genes in endometriotic lesions showed that expression of candidate factors (Il4, Cxcl15, Il8, Il21, Tnf, Il4ra, Cxcr1, Cxcr2, Il18r, Il21r, and Tnfrsfa) was significantly reduced compared to the control group (FIGS.

2a, 2b). Under the same conditions, fraxetin also inhibited the expression of adhesion-related genes (Icam1, Vcam1, Itga1, Itga5), increased the expression of apoptosis-related genes (Bak1, Bax, Bad, Casp1), increased the expression of endoplasmic reticulum stress-related genes (Hspa5, Elf2s1, Ern1), and decreased the expression of mitochondrial function-related genes (Atp5me, Atp5pb, Atp5po).

These results indicate that fraxetin may inhibit the development of endometriotic lesions by regulating the expression of inflammation-related, adhesion-related, apoptosis-related, endoplasmic reticulum stress-related, and mitochondrial function-related genes.

### EXAMPLE 2: Inhibitory Effect of Fraxetin on Cell Proliferation in Human Endometrial Cell Lines

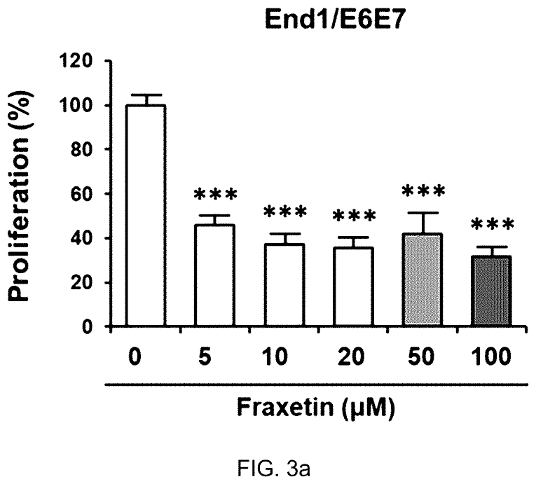

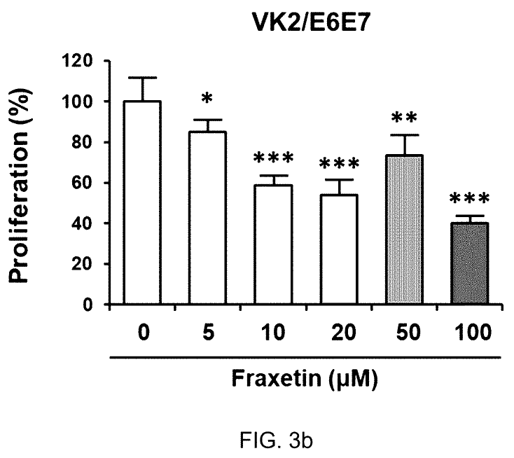

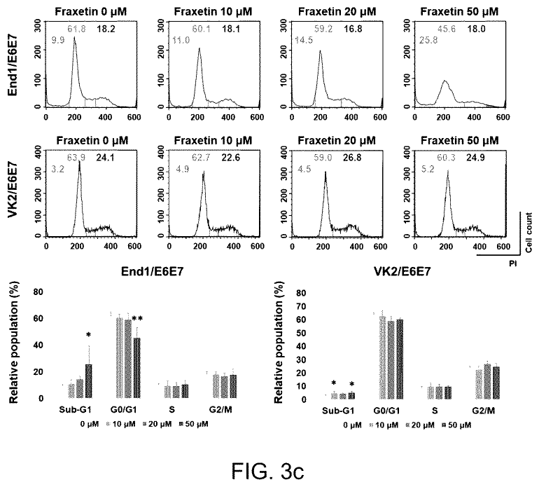

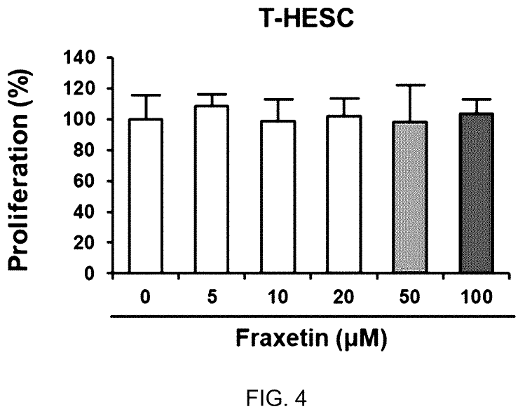

To examine the effect of fraxetin on endometrial cell proliferation, the inventors conducted BrdU cell proliferation assays and cell cycle analysis, with results shown in FIGS. 3 and 4.

According to FIGS. 3a and 3b, fraxetin treatment reduced cell proliferation to below 50% at all concentrations in End1/E6E7 cells and reduced proliferation by approximately 40% at 100 μM in VK2/E6E7 cells. However, as shown in FIG. 4, no changes in cell proliferation were observed in normal endometrial cells treated with fraxetin.

Cell cycle analysis showed an increase in the number of cells in the Sub-G1 phase, indicating apoptosis in both endometrial cell lines (FIG. 3c).

These results suggest that fraxetin may inhibit cell proliferation in endometrial cell lines and regulate the cell cycle, thereby supporting its potential use in endometriosis treatment.

### EXAMPLE 3: Analysis of Cell Migration and Inhibition of MAPK/AKT Signaling Pathways in Human Endometrial Cells Treated with Fraxetin

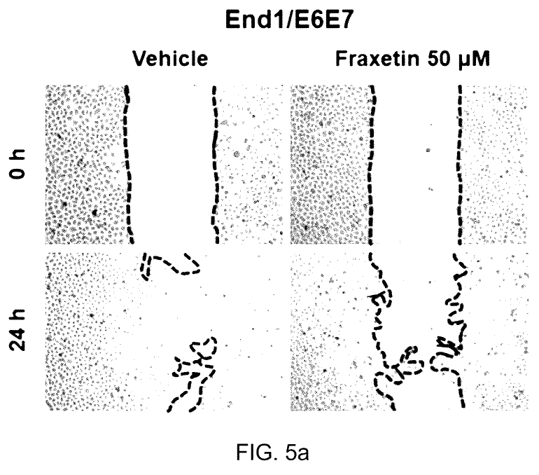

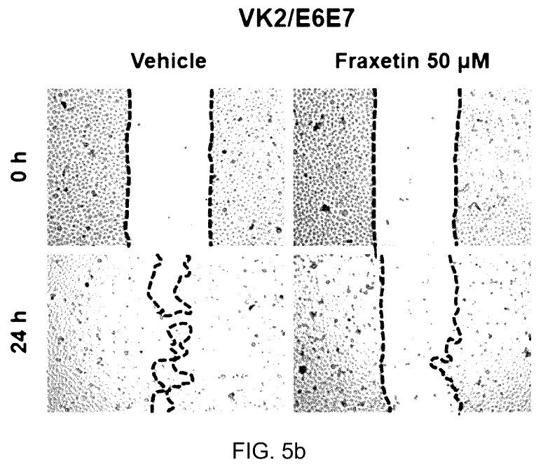

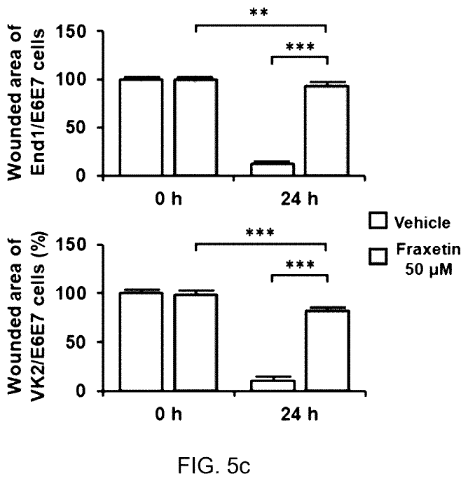

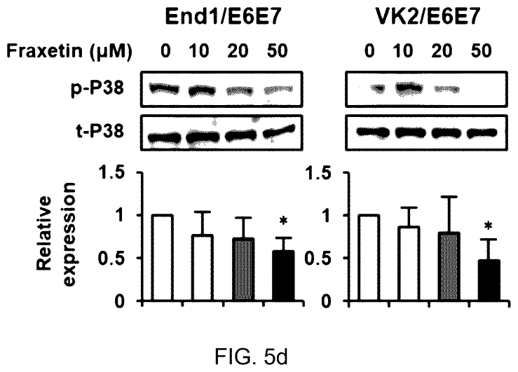

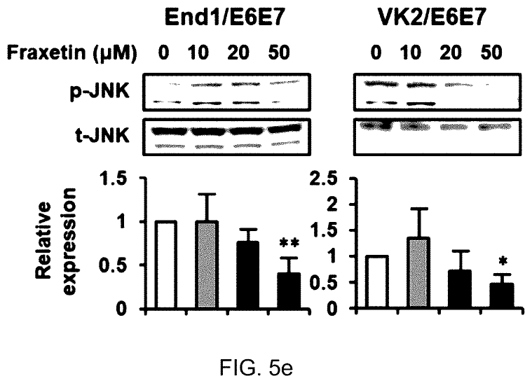

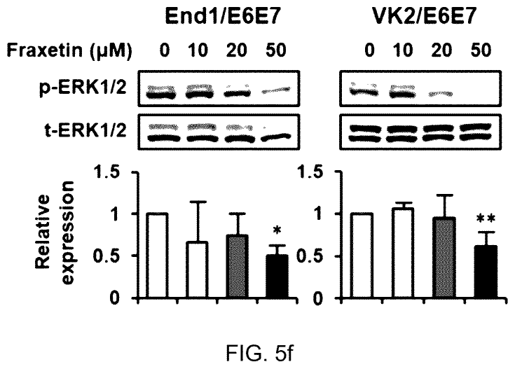

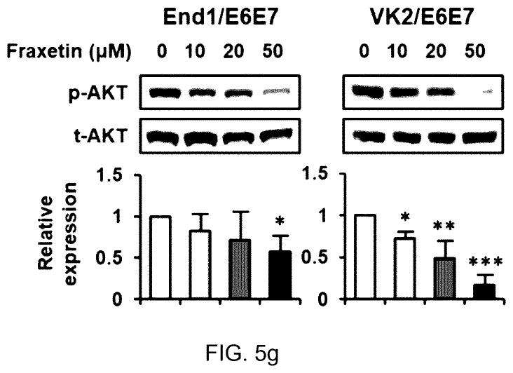

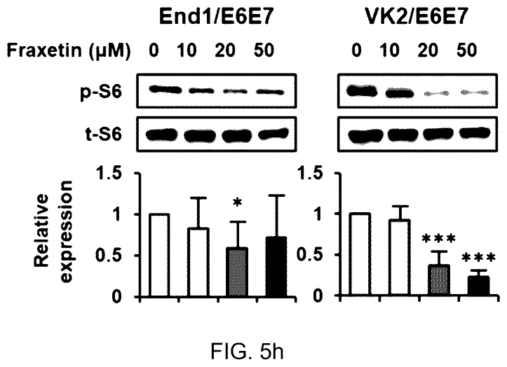

To investigate the effect of fraxetin on cell migration in endometrial cells, the inventors conducted a wound healing assay, with results shown in FIGS. 5a to 5c.

In the End1/E6E7 cells, as shown in FIGS. 5a and 5c, the DMSO-treated group (Vehicle) exhibited 88% wound closure, while the group treated with 50 μM fraxetin showed only 7% closure. Similarly, as shown in FIGS. 5b and 5c, in VK2/E6E7 cells, the DMSO-treated group (Vehicle) exhibited 90% wound closure, while the group treated with fraxetin showed only 18% closure.

Additionally, western blot analysis was performed to observe changes in the expression of proteins related to cell proliferation, survival, and migration, with results shown in FIGS. 5d to 5h.

Upon treatment with 50 μM fraxetin, p38phosphorylation was reduced by 0.61-fold and 0.47-fold compared to the control group (FIG. 5d), and JNK phosphorylation was inhibited to below 50% (FIG. 5e). ERK1/2 phosphorylation was gradually inhibited to 0.51-fold and 0.61-fold, respectively, in the 50 μM fraxetin-treated group (FIG. 5f), and AKT phosphorylation was inhibited by 57% compared to the control, while S6 phosphorylation was reduced by 59% upon treatment with 20 μM fraxetin in End1/E6E7 cells (FIGS. 5g and 5h).

These results suggest that fraxetin may help treat endometriosis by regulating cell migration through modulation of MAPK/AKT signaling pathway proteins.

### EXAMPLE 4: Analysis of Cell Proliferation Signaling Pathways in Endometrial Cells Treated With Fraxetin Under MAPK/AKT Inhibitor Conditions

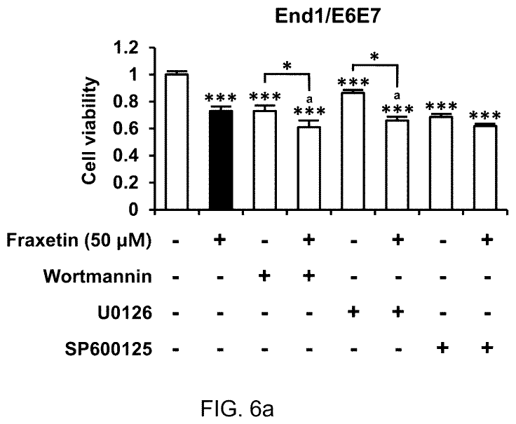

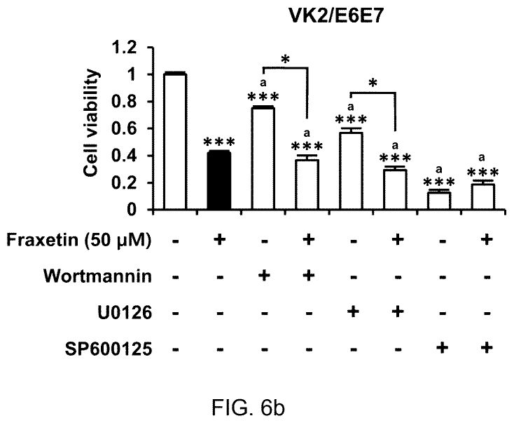

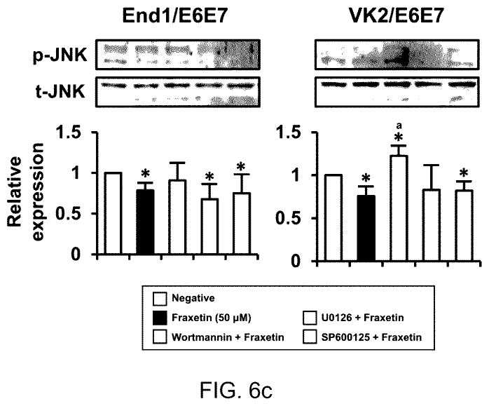

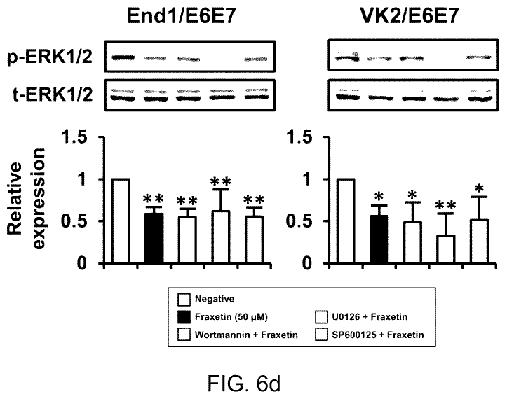

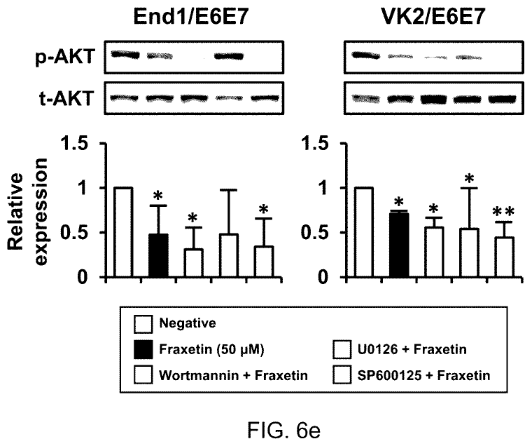

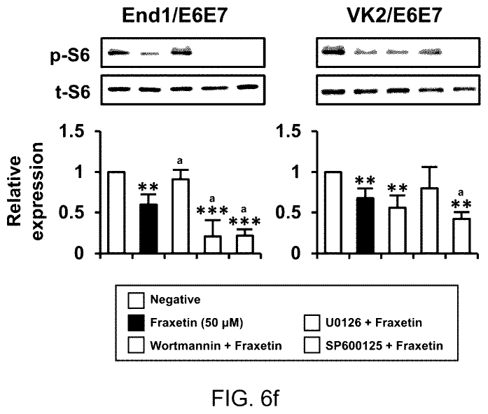

The inventors further investigated whether fraxetin's downregulation of the MAPK and AKT pathways is associated with the attenuation of endometriosis. Western blot analysis was conducted to observe phosphorylation patterns of proteins in the PI3K/AKT and MAPK signaling pathways related to cell proliferation under conditions of single or combined treatment with signaling pathway inhibitors, with results shown in FIG. 6.

The AKT inhibitor wortmannin (1 μM), ERK1/2 inhibitor U0126 (20 μM), and JNK inhibitor SP600125 (20 μM) were used.

As shown in FIGS. 6a and 6b, all inhibitors significantly suppressed the viability of both types of endometrial cells. In particular, combined treatment of wortmannin or U0126 with fraxetin showed a marked decrease in cell viability in both End1/E6E7 and VK2/E6E7 cells compared to treatment with each pharmacological inhibitor alone.

Additionally, western blot analysis results in FIGS. 6c to 6f showed that wortmannin treatment had no significant effect on JNK phosphorylation in End1/E6E7 cells, but increased JNK phosphorylation was observed in VK2/E6E7 cells, suggesting that AKT suppresses JNK and that other inhibitors also showed downregulation (FIG. 6c). For ERK1/2 and AKT phosphorylation, significant downregulation was observed upon combined treatment with inhibitors, although no synergistic effect was noted. However, compared to fraxetin alone, U0126 and SP600125 further downregulated S6 phosphorylation in End1/E6E7 cells, and SP600125 further downregulated S6 phosphorylation in VK2/E6E7 cells (FIG. 6f).

These results suggest that combined treatment of fraxetin and signaling pathway inhibitors significantly downregulates signaling pathway proteins, effectively inhibiting endometriosis compared to fraxetin treatment alone.

### EXAMPLE 5: Analysis of Mitochondrial and Homeostasis Regulation Effects in Human Endometrial Cells Treated With Fraxetin

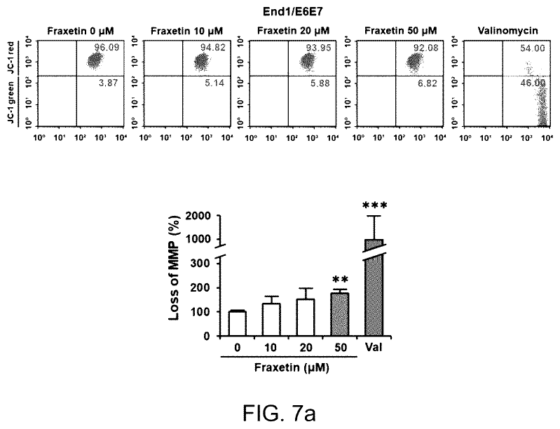

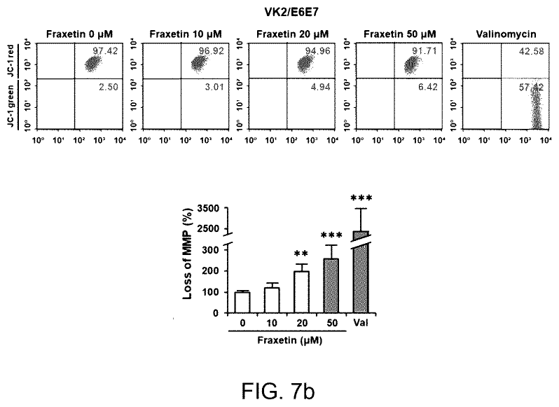

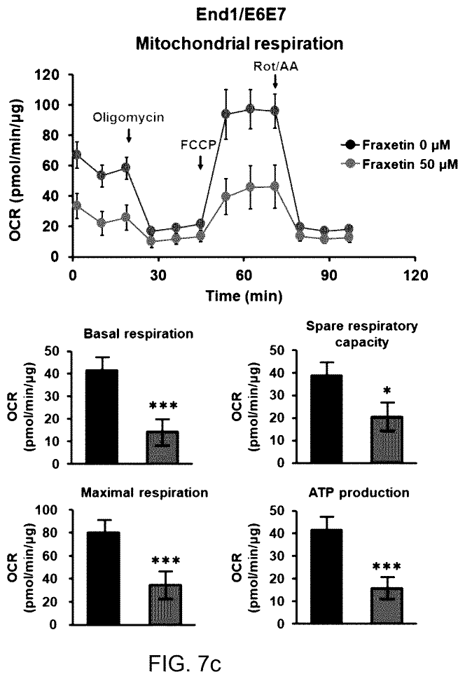

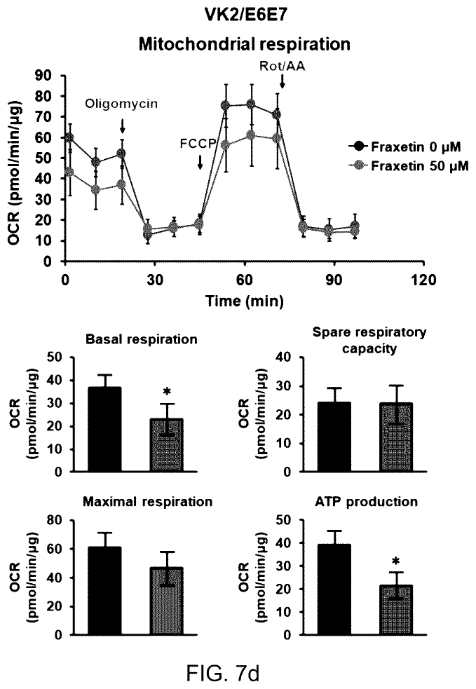

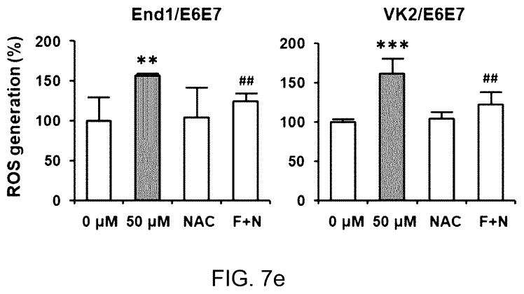

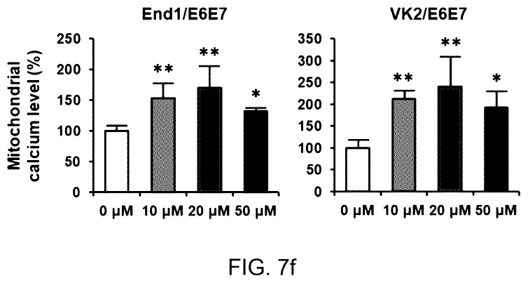

To examine the effect of fraxetin on mitochondrial function, the inventors measured bioenergetics (ATP), calcium levels, and redox homeostasis, with results shown in FIG. 7.

Fraxetin treatment disrupted the high electrochemical gradient in mitochondria, resulting in a relative membrane potential loss rate increase of 1.8-fold in End1/E6E7 cells and 2.6-fold in VK2/E6E7 cells (FIGS. 7a and 7b).

Additionally, as shown in FIGS. 7c and 7d, basal and maximal respiration and ATP production in End1/E6E7 cells significantly decreased, with reserve respiratory capacity halved compared to the control group. In VK2/E6E7 cells, only basal respiration and ATP production decreased.

Next, to assess the effect of fraxetin on reactive oxygen species (ROS) generation, ROS levels were measured in fraxetin-treated endometrial cells, with and without the ROS inhibitor NAC. As shown in FIG. 7e, ROS generation increased by up to 1.5-fold in fraxetin-treated End1/E6E7 and VK2/E6E7 cells compared to the control group, and this increase was mitigated by co-treatment with 2 mM NAC in both cell lines.

Finally, intracellular mitochondrial calcium ion concentration was analyzed in fraxetin-treated endometrial cells, and as shown in FIG. 7f, calcium levels significantly increased to 1.7-fold and 2.4-fold in cells treated with 20 μM fraxetin, with a slight increase observed in cells treated with 50 μM fraxetin.

These results suggest that fraxetin may exert potential therapeutic effects on pathological conditions associated with endometriosis by regulating mitochondrial function in endometrial cells.

### EXAMPLE 6: Analysis of ER Stress Activation and Mitochondria-Mediated Apoptosis in Human Endometrial Cells Treated with Fraxetin

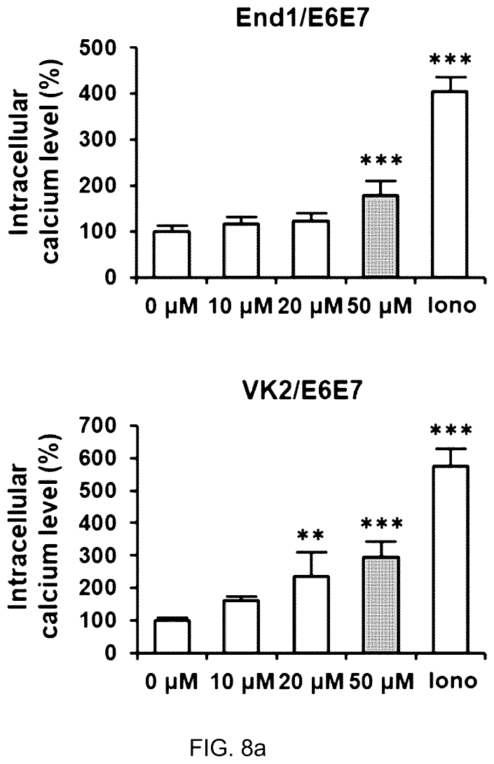

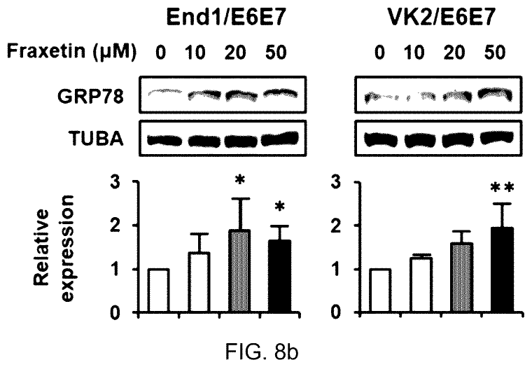

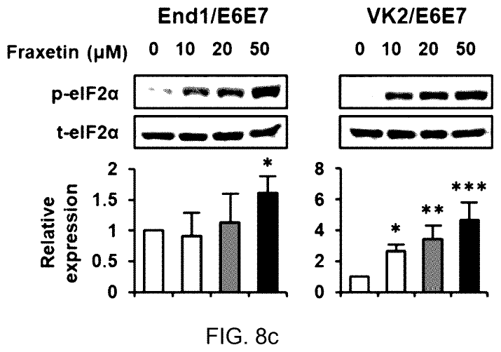

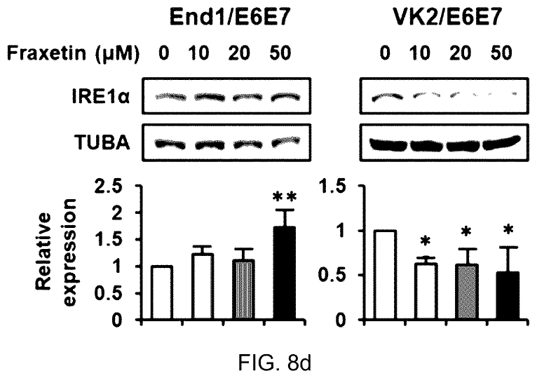

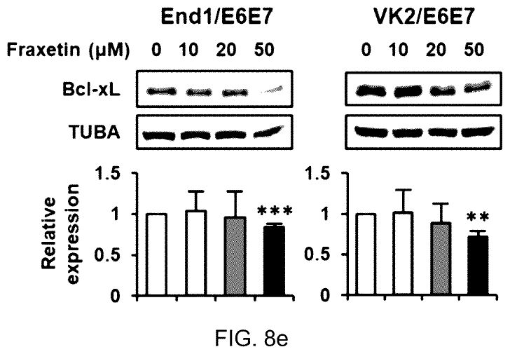

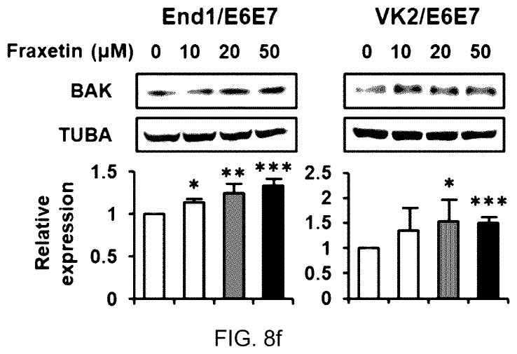

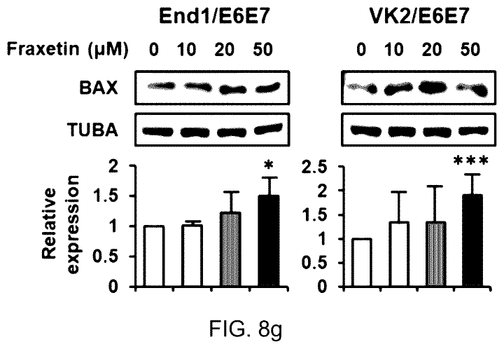

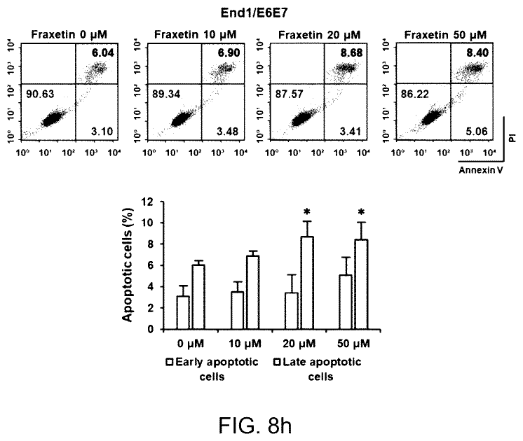

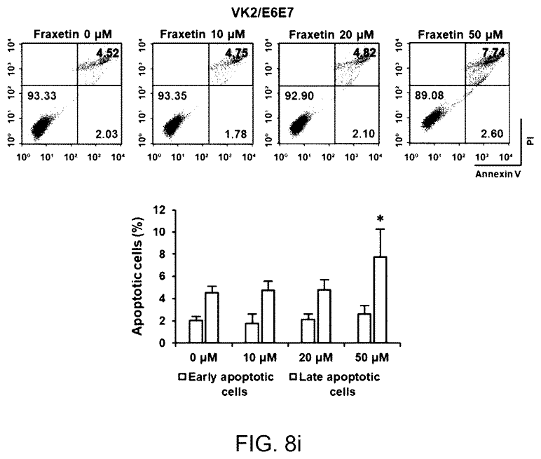

The inventors examined the effects of fraxetin on ER stress and mitochondria-mediated apoptosis in endometrial cells, with results shown in FIG. 8.

As shown in FIG. 8a, fraxetin treatment upregulated calcium levels in End1/E6E7 and VK2/E6E7 cells to 180% and 300%, respectively.

Next, phosphorylation patterns of ER stress response factors were examined using western blot analysis, with results shown in FIGS. 8b and 8g.

The level of GRP78, an initiator of the UPR, increased 1.9-fold in cells treated with 20 μM and 50 μM fraxetin compared to the control, and eIF2α phosphorylation also tended to increase, suggesting that fraxetin treatment may block translation initiation and protein synthesis.

In contrast, IRE1α expression increased 1.7-fold in End1/E6E7 cells but decreased to 0.53-fold in VK2/E6E7 cells (FIG. 8d).

Regarding mitochondrial-related proteins, fraxetin treatment reduced the expression of the anti-apoptotic protein Bcl-xL in both End1/E6E7 and VK2/E6E7 cells (FIG. 8e), while pro-apoptotic proteins BAK and BAX were increased (FIGS. 8f and 8g).

Finally, both early and late apoptosis cell populations tended to increase in fraxetin-treated endometrial cell lines.

These results suggest that fraxetin-induced apoptosis is mediated by mitochondrial dysfunction and ER stress activation, indicating that fraxetin may be useful for treating endometriosis.

### EXAMPLE 7: Analysis of Reactive Oxygen Species (ROS) Generation and Apoptosis Induction by Dienogest and Fraxetin in Endometriosis Treatment

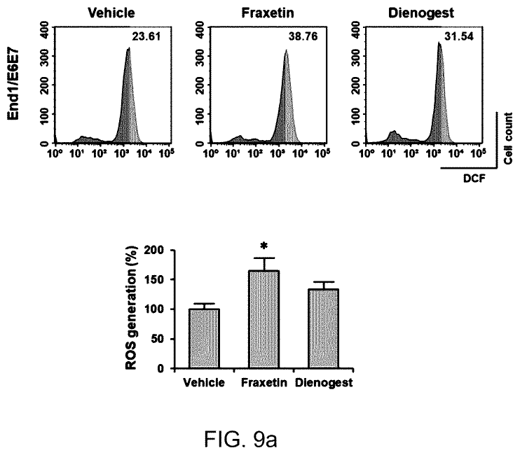

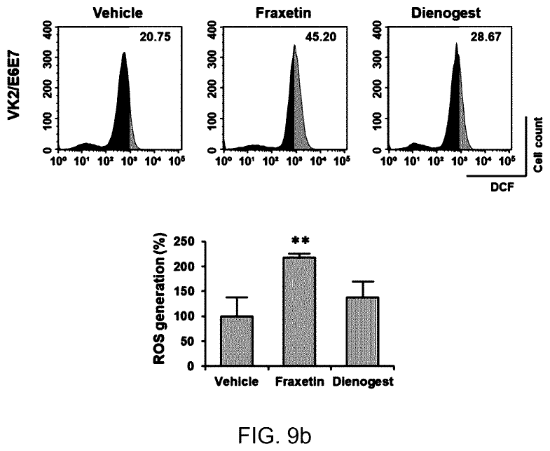

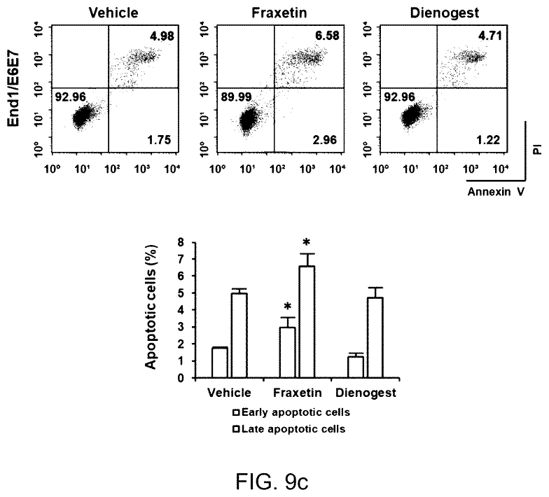

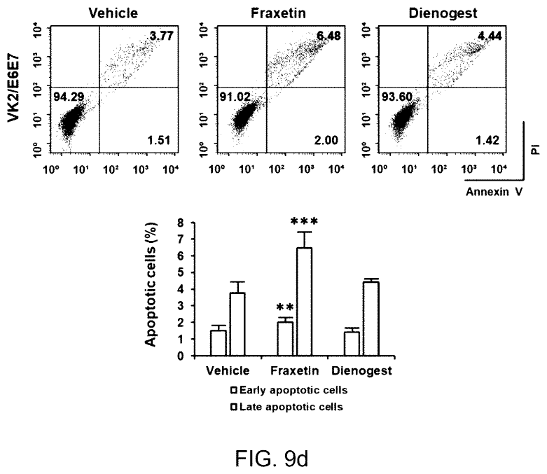

To compare the efficacy of fraxetin with that of an existing treatment, the inventors investigated ROS generation and apoptosis patterns following treatment with fraxetin and dienogest. The results are shown in FIG. 9.

As shown in FIGS. 9a and 9b, fraxetin significantly increased relative ROS generation by over 150% and 200% in End1/E6E7 and VK2/E6E7 cells, respectively, while dienogest did not show significant changes in either cell type.

Additionally, unlike dienogest, fraxetin induced both early and late apoptosis in endometrial cells.

These results indicate that fraxetin induces relatively higher ROS generation and apoptosis in endometrial cells compared to dienogest, an existing treatment.

### EXAMPLE 8: Analysis of the Effects of Fraxetin-Mediated tiRNA Regulation on Cell Proliferation, Mitochondrial Function, and Apoptosis in Endometriosis Cells

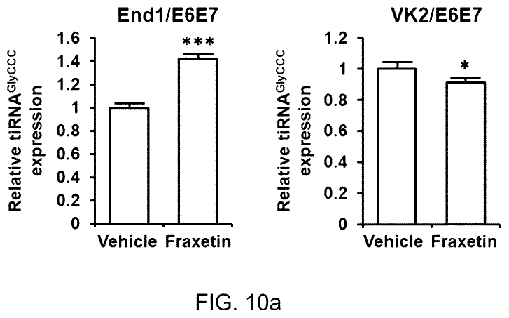

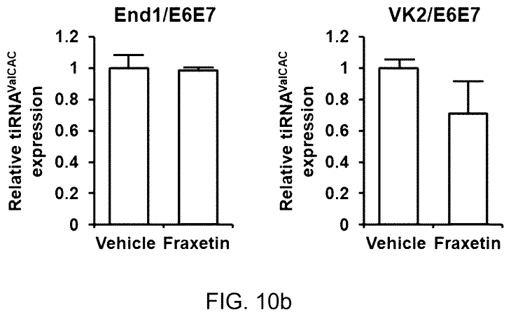

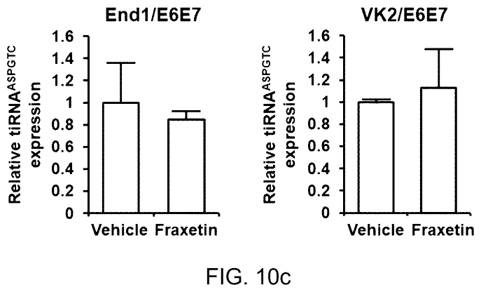

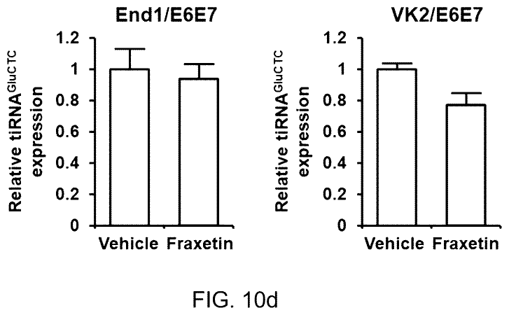

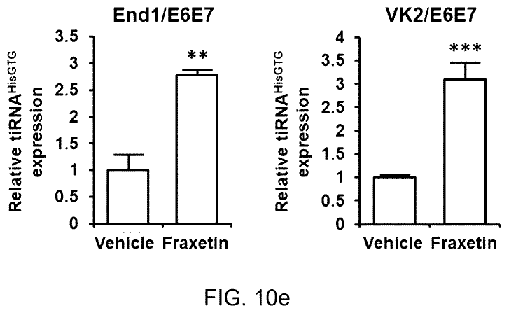

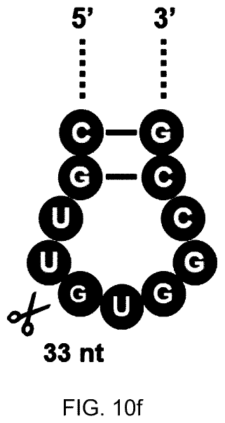

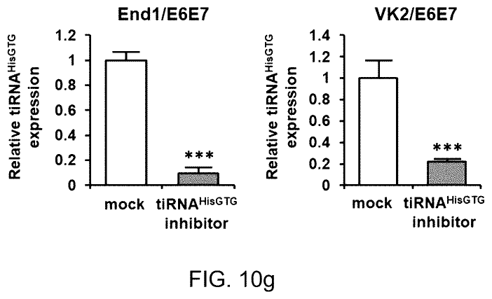

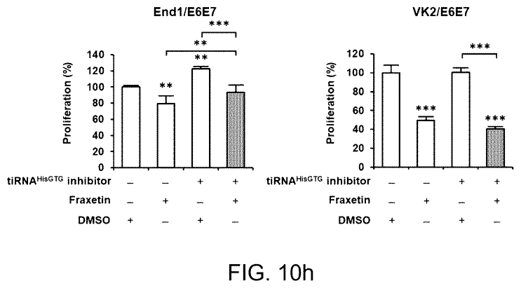

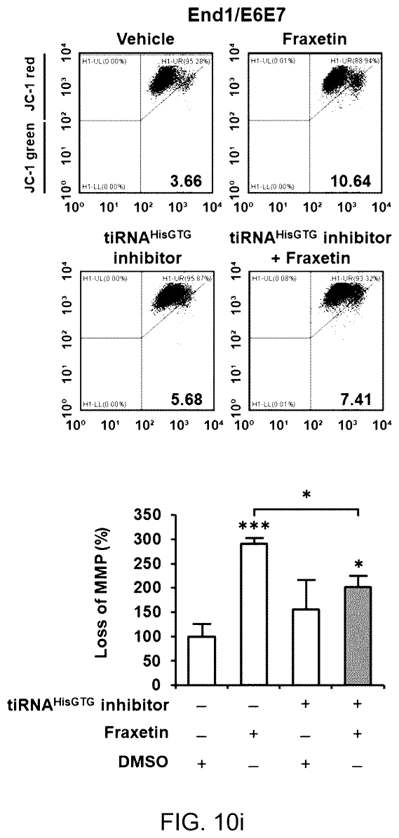

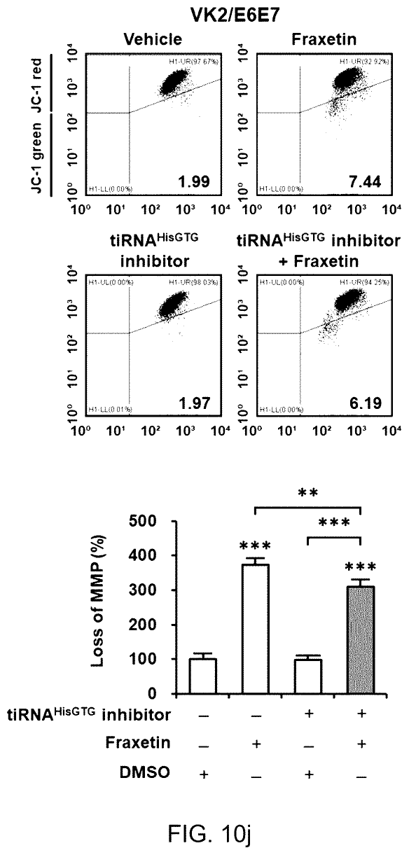

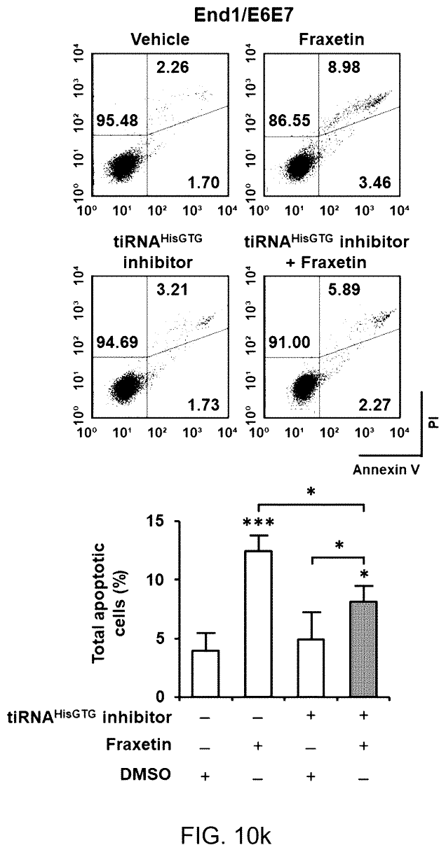

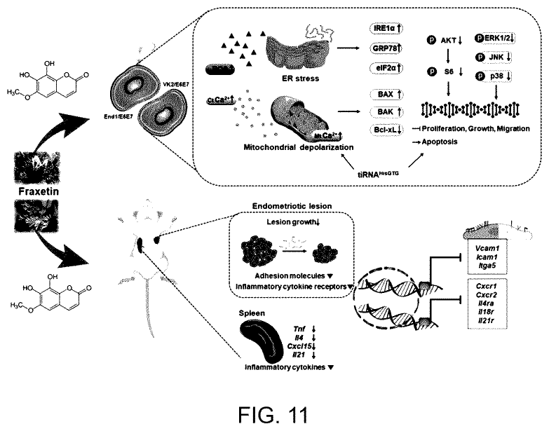

The inventors selected candidate tiRNAs associated with the development of endometriosis and investigated the effect of fraxetin on the expression of these selected tiRNA candidates, with results shown in FIG. 10.

For tiRNA^{GlyCCC}, tiRNA^{ValCAC}, tiRNA^{AspGTC}, and tiRNA^{GluCTC}, there was minimal change in mRNA expression following fraxetin treatment (FIGS. 10a to 10d), but tiRNA^{HisGTG} expression increased by more than twofold (FIG. 10e).

Next, cell proliferation changes under fraxetin treatment were analyzed with tiRNA^{HisGTG} inhibitors. In both cell lines, tiRNA^{HisGTG} mRNA expression decreased upon the introduction of the tiRNA^{HisGTG} inhibitor (FIG. 10g). Additionally, when cells were sequentially treated with the tiRNA^{HisGTG} inhibitory gene and fraxetin for 48 hours, cell proliferation decreased (FIG. 10h).

Finally, to confirm the functional role of tiRNA^{HisGTG}, mitochondrial membrane potential (MMP) changes and apoptosis were examined following tiRNA^{HisGTG} inhibitor treatment. In both endometriosis cell lines, fraxetin-induced MMP loss was significantly mitigated by tiRNA^{HisGTG} inhibitor treatment (FIGS. 10i, j), and fraxetin-induced apoptosis was inhibited (FIGS. 10k, l). These results suggest that fraxetin regulates tiRNA^{HisGTG}, which is involved in the development and progression of endometriosis, providing a novel strategy for endometriosis treatment.

What is claimed is:

1. A method for preventing or treating endometriosis and a complication associated therewith, the method comprising a step of administering fraxetin, represented by Chemical Formula 1, or a pharmaceutically acceptable salt thereof, to a subject in need thereof:

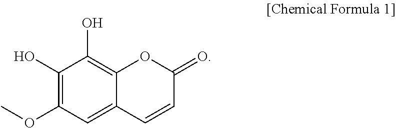

2. The method of claim 1, wherein the fraxetin or a pharmaceutically acceptable salt thereof inhibits proliferation of endometrial cells.

3. The method of claim 1, wherein the fraxetin or a pharmaceutically acceptable salt thereof induces mitochondrial dysfunction in endometrial cells.

4. The method of claim 1, wherein the fraxetin or a pharmaceutically acceptable salt thereof increases endoplasmic reticulum stress in endometrial cells.

5. The method of claim 1, wherein the endometriosis-associated complication is at least one selected from the group consisting of pelvic inflammatory disease, pelvic adhesions, ovarian cysts, uterine fibroids, ectopic pregnancy, and infertility.

6. The method of claim 1, further comprising a step of administering a PI3K/AKT signaling pathway inhibitor, MAPK signaling pathway inhibitor, or GnRH agonist to the subject in need thereof.

7. The method of claim 6, wherein the PI3K/AKT signaling pathway inhibitor is wortmannin.

8. The method of to claim 6, wherein the MAPK signaling pathway inhibitor is U0126.

9. The method of claim 6, wherein the GnRH agonist is dienogest.
